(19) United States
(12) Patent Application Publication
(10) Pub. No.: US 2005/0042608 A1
(43) Pub. Date: Feb. 24, 2005

(54) Detection of single nucleotide polymorphisms

(75) Inventor: Allen E. Eckhardt, Durham, NC (US)

Correspondence Address: DORSEY & WHITNEY LLP;INTELLECTUAL PROPERTY DEPARTMENT, 4 EMBARCADERO CENTER, SUITE 3400, SAN FRANCISCO, CA, 94111, US

(73) Assignee: Clinical Micro Sensors, Inc.

(21) Appl. No.: 10/479,751

(22) PCT Filed: May 31, 2002
(86) PCT No.: PCT/US02/16993

Related U.S. Application Data
(63) Continuation-in-part of application No. 09/873,809, filed on Jun. 4, 2001, now abandoned.

(51) Int. Cl. C12Q 1/68

(52) U.S. Cl. 435/6

(57) ABSTRACT

A method determining the presence or absence of a single nucleotide polymorphism at a SNP site in a nucleic acid target. Capture probes are designed, each of which has a different SNP base and a sequence of probe bases on each side of the SNP base. The probe bases are complementary to the corresponding target sequence adjacent to the SNP site. Each capture probe is immobilized on a different electrode having a non-conductive outer layer on a conductive working surface of a substrate. The extent of hybridization between each capture probe and the nucleic acid target is detected by detecting the oxidation-reduction reaction at each electrode, utilizing a transition metal complex. These differences in the oxidation rates at the different electrodes are used to determine whether the selected nucleic acid target has a single nucleotide polymorphism at the selected SNP site.

### BACKGROUND OF THE INVENTION

1. Field of the Invention

This invention relates to a method of determining single nucleotide polymorphisms.

2. Description of the Related Art

Single Nucleotide Polymorphism Studies. A single nucleotide polymorphism (SNP) is a single base change or point mutation resulting in genetic variation between individuals. SNPs occur in the human genome approximately once for every kilobase of the genome, and can occur in coding or non-coding regions of the genome. A SNP in the coding region may or may not change the amino acid sequence of a protein product. A SNP in a non-coding region can alter promoters or processing sites and affect gene transcription and processing.

Knowledge of whether an individual has a particular SNP may provide sufficient information to develop diagnostic, preventative and therapeutic applications for a variety of diseases. In particular, such information allows prediction of drug responses, selection of clinical trial subjects, and identification of genetic subgroups, such as those susceptible to drug side-effects. A number of databases have been constructed of known SNPs and the effect of a SNP at a particular site. Examples of genetic conditions that are related to SNPs include sickle-cell anemia and long QT syndrome for sudden death from ventricular tachyarrhythmias.

Other general information on SNPs is found in Wang et al. (1998, Science 280, 1077-1082); and Collins et al. (1997, Science 278, 1580-1581). Rutter et al. (1998, Cancer Research 58, 5321-5325) discusses the impact a particular SNP has in a defined biochemical system. The disclosure of all patents and publications referred to herein is incorporated herein by reference.

A wide variety of techniques have been devised to determine whether a particular genome or gene has a particular SNP present. Generally, unlike the instant invention, these prior techniques require fluorescent or enzymatic labeling.

Some of these techniques require that the target material be amplified using a method such as the polymerase chain reaction (PCR). For example, the amplification of gene sequences has enabled sequencing of particular PCR products, as in the products of PE Biosystems (Foster City, Calif.). The specific hybridization of PCR primers to either wild-type or mutant alleles in PCR products enables accumulation of evidence on the genotype being investigated (AndCare, Inc., Durham, N.C., and Thetagen, Inc., Bothell, Wash.).

Another technique for determining SNPs includes use of the mass spectrometer to measure probes that hybridize to the SNP. This technique varies in how rapidly it can be performed, from a few samples per day to a high throughput of 40,000 SNPs per day, using mass code tags. Companies using such techniques include Rapigene Inc. (Bothell, Wash.), Perseptive Biosystems (Foster City, Calif.) and Orchid Biocomputer (Princeton, N.J.). See Ross et al., 1997, Anal. Chem. 69, 4197-4202.

SNPs can also be determined by ligation-bit analysis. This analysis requires two primers that hybridize to a target with a one nucleotide gap between the primers. Each of the four nucleotides is added to a separate reaction mixture containing DNA polymerase, ligase, target DNA and the primers. The polymerase adds a nucleotide to the 3′end of the first primer that is complementary to the SNP, and the ligase then ligates the two adjacent primers together. Upon heating of the sample, if ligation has occurred, the now larger primer will remain hybridized and a signal, for example, fluorescence, can be detected. A further discussion of these methods can be found in U.S. Pat. Nos. 5,919,626; 5,945,283; 5,242,794; and 5,952,174.

The techniques of Affymetrix (Santa Clara, Calif.) and Nanogen Inc. (San Diego, Calif.) utilize the fact that DNA duplexes containing single base mismatches are much less stable than duplexes that are perfectly base-paired. The presence of a matched duplex is detected by fluorescence.

Another fluorescent technique for SNP analysis involves allowing a primer to hybridize to the DNA sequence adjacent to the SNP site on the test sample under investigation. The primer is extended by one nucleotide using all four differentially tagged fluorescent dideoxynucleotides (A,C,G, or T), and a DNA polymerase. Only one of the four nucleotides (homozygous case) or two of the four nucleotides (heterozygous case) is incorporated. The base that is incorporated is complementary to the nucleotide at the SNP position. This technique is used by Packard Instrument Company (Meriden, Conn.), PE Biosystems (Foster City, Calif.) and Orchid Biocomputer Inc. (Princeton, N.J.).

The technique of Lynx Therapeutics (Hayward, Calif.) using MEGATYPE™ technology can genotype very large numbers of SNPs simultaneously from small or large pools of genomic material. This technology uses fluorescently labeled probes and compares the collected genomes of two populations, enabling detection and recovery of DNA fragments spanning SNPs that distinguish the two populations, without requiring prior SNP mapping or knowledge.

Finally, other techniques rely on conformational differences between molecules. PCR products amplified with the same primers but containing different SNPs will have a different conformation after denaturing and annealing. This change in conformation results in a mobility shift in non-denaturing acrylamide gels that can be used to differentiate SNPs, relying on single-stranded conformational polymorphism (SSCP).

The need to amplify and label the sample, and the difficulty of performing large numbers of analyses in the prior methods for SNP determination mean that these techniques are generally more time-intensive or labor-intensive than is desirable.

Electrochemical Detection of Nucleic Acid Hybridization. The invention herein utilizes the prior method of electrochemical detection of nucleic acid hybridization of Thorp et al. (U.S. Pat. No. 5,871,918), the disclosure of which patent is incorporated herein by reference. Briefly, this patent discloses a new method of sequencing, and methods of qualitatively and quantitatively detecting a nucleic acid, such as DNA or RNA, that contains at least one preselected base, for example, adenine, guanine, 6-mercaptoguanine, 8-oxo-guanine, 8-oxo-adenine, or other base that undergoes oxidation upon reaction with a selected oxidizing agent. The method of Thorp et al. comprises: a) reacting the nucleic acid with a transition metal complex (mediator) capable of oxidizing the preselected base in an oxidation-reduction reaction; b) detecting the oxidation-reduction reaction; and c) determining the presence or absence of the nucleic acid from the detected oxidation-reduction reaction at the preselected base.

Depending on the particular embodiment of the method of Thorp et al. that is employed, the method of Thorp et al. may optionally include the step of contacting the nucleic acid with a complementary nucleic acid probe to form a hybridized nucleic acid, generally as an initial step. The probe used in the method of Thorp et al. may be from about 4-6 bases to about 100 bases or more, and preferably is about 12-30 bases. The nucleic acid being analyzed may optionally be amplified using methods known in the art prior to contacting with the nucleic acid probe.

A preferred transition metal complex for use as an oxidizing agent in the electrochemical detection of Thorp et al., which is reactive with the preselected base at a unique oxidation potential, is ruthenium²⁺ (2,2′-bipyridine)₃ (Ru(bpy)₃²⁺). Other suitable transition metal complexes are disclosed in U.S. Pat. No. 5,871,918, the disclosure of which is incorporated herein.

The detection of the oxidation-reduction reaction of Thorp et al. typically utilizes a detection electrode that is sensitive to the transfer of electrons between the oxidizing agent (the transition metal complex) and the hybridized nucleic acid. Such an electrode is placed in contact with the solution containing the reacted hybridized nucleic acid and the oxidizing agent, along with a reference electrode and an auxiliary electrode. Suitable electrodes are known in the art, with a preferred electrode being an indium tin oxide electrode. The step of determining the presence or absence of hybridized nucleic acid typically includes (i) measuring the reaction rate of the oxidation-reduction reaction; (ii) comparing the measured reaction rate to the oxidation-reduction reaction rate of the transition metal complex with a single-stranded nucleic acid; and (iii) determining whether the measured reaction rate is essentially the same as the oxidation-reduction rate of the transition metal complex with the single-stranded nucleic acid.

The oxidation-reduction rate may be determined by comparing the current as a function of scan rate, probe concentration, target concentration, transition metal complex, buffer, temperature, and/or electrochemical method. Typically, the oxidation-reduction reaction rate in the Thorp method is measured by measuring the electronic signal associated with the occurrence of the oxidation-reduction reaction, for example, by providing a suitable apparatus (e.g., a potentiostat) in electronic communication with the detection electrode, using methods such as cyclic voltammetry, normal pulse voltammetry, chronoamperometry, and square-wave voltammetry. Cyclic voltammetry and chronoamperometry are the preferred methods. The patent of Thorp et al. also teaches various types of apparatus, electrode structures and microelectronic devices for carrying out the nucleic acid detection.

It is therefore an object of the invention to provide a method of SNP determination, utilizing a method of Thorp et al., that is both simple and sensitive, and that can rapidly be performed on many samples, because it does not require amplification of genomic material, and can be performed using a small volume of sample. In addition, the method of the invention does not generally require purification of the target.

It is also an object of the invention to provide a method of SNP determination using a straightforward interrogation technique that provides a simple yes or no qualitative result that is universal in its application.

Other objects and advantages will be more fully apparent from the following disclosure and appended claims.

### SUMMARY OF THE INVENTION

The invention herein is a method of SNP determination comprising a method of determining the presence or absence of a single nucleotide polymorphism at a selected site (the “SNP site”) in a selected nucleic acid target. Using information about the normal base composition of the target at the selected SNP site and adjacent to the selected SNP site, capture probes are designed, each of which capture probes has a SNP base and a sequence of probe bases on each side of the SNP base. Preferably, there are about 9-31 nucleotides in the capture probe. The probe bases on each side of the SNP base are complementary to the corresponding target sequence adjacent to the selected SNP site. The SNP base is different in each of the capture probes that are being used to determine whether there is a single nucleotide polymorphism at the SNP site in the target nucleic acid. Each capture probe is immobilized on a different electrode having a non-conductive outer layer on a conductive working surface of a substrate. The extent of hybridization between each capture probe and the nucleic acid target is detected by detecting the oxidation-reduction reaction at each electrode, utilizing a transition metal complex. There will be different oxidation-reduction rates at the different electrodes depending on whether the nucleic acid target has hybridized to the capture probe. These differences in the oxidation rates at the different electrodes are used to determine whether the selected nucleic acid target has a single nucleotide polymorphism at the selected SNP site.

Other objects and features of the inventions will be more fully apparent from the following disclosure and appended claims.

### BRIEF DESCRIPTION OF THE DRAWINGS

FIG. 1 is a schematic diagram of the use of the preferred method of the invention, showing how the capture probes would be structured.

FIG. 2 is a schematic diagram of the use of the preferred method of the invention, showing attachment of target nucleic acid to one of the capture probes.

FIG. 3 is a schematic diagram of the use of a sandwich assay in the method of the invention.

### DETAILED DESCRIPTION OF THE INVENTION AND PREFERRED EMBODIMENTS THEREOF

The present invention provides a method of determination of the presence of SNPs (single nucleotide polymorphisms) in one or more samples, using capture probes, and an oxidizing agent, such as a suitable transition metal complex.

Oxidizing Agents and Oxidation-Reduction Reactions. The preferred transition metal complex for use as a mediator in the methods of the present invention is Ruthenium²⁺ (2,2′-bipyridine)₃ (“Ru(bpy)₃²⁺ ”). Alternatively, other transition metal complexes that have a mid-point oxidation-reduction potential of approximately 0.9 volts or greater may be suitable for use. Some anionic complexes and zwitterionic complexes, in addition to transitional metal complexes having suitable substituted derivatives of the pyridine, bypyridine and phenanthroline groups, may also be employed, provided that the selected complex oxidizes the target in an oxidation-reduction reaction so that there is electron transfer from the target to the complex, resulting in regeneration of the reduced form of the complex as part of a catalytic cycle.

Probes. The capture probes of the invention comprise polymers of a) selected bases; and b) a backbone as discussed below. The probes of the invention have any of a wide variety of base sequences and may be prepared according to techniques which are well known in the art, so long as they possess a base sequence at least a portion of which is capable of binding to the nucleotides adjacent to the SNP site of the sample nucleic acid.

The bases of the capture probe include a SNP base and a plurality of probe bases on each side of the SNP base. Generally, “probe bases”, as used herein and discussed more specifically below, are selected from the naturally occurring nucleotide bases, adenine, cytosine, guanine, and thymine. As discussed in more detail below, guanine's presence in the target is the basis for detecting the difference in current at the different electrodes. Therefore, in selecting the bases for the probe, it is desirable to minimize the signal from the guanines in the probe. Thus, alternate bases that will not oxidize under the same conditions as guanine, but will still hybridize with cytosine may be substituted for guanine in the probe. An example of this is inosine. The “SNP bases” that are at a location of the probe corresponding to the SNP site are adenine, cytosine, guanine, or thymine.

The backbone of the capture probe is preferably a neutral backbone, for example, a peptide backbone, a p-ethoxy backbone or a morpholine backbone. Thus, the resultant capture probe including the backbone and the bases is preferably a peptide nucleic acid (PNA) such as is described in P. Nielsen et al., 1991, Science 254, 1497-1500, a p-ethoxy nucleic acid, a morpholino nucleic acid or other base-containing probe with a neutral backbone species.

The backbone may also be a sugar-phosphate or any other moiety that connects the bases and supports hybridization of the capture probe. Such probes are then, for example, DNA, RNA or any other nucleic acid analog. Thus, backbones including modified sugars such as carbocycles, and sugars containing 2′ substitutions such as fluoro and methoxy are included within the capture probes of the invention. The oligonucleotides may be oligonucteotides wherein at least one, or all, of the internucleotide bridging phosphate residues are modified phosphates, such as methyl phosphonates, methyl phosphonothioates, phosphoromorpholidates, phosphoropiperazidates and phosphoramidates (for example, every other one of the internucleotide bridging phosphate residues may be modified as described). The only requirement is that the probe should possess a sequence which is complementary to a known portion of the sequence of the target nucleic acid.

The capture probes of the invention must be long enough to provide both functional binding and specificity for the SNP target, and which depends on the number and sequence of probe bases used. The probe size is preferably from 9-31-mers (i.e., 9-31 bases including the SNP base) with a sequence of probe bases flanking both sides of the SNP base; however, the preferred range may differ depending on the backbone. A short probe length enables magnification of the impact of a mismatch at the site of the polymorphism; however, too short a probe may hybridize to other areas and complicate the analysis. Thus, using a probe 13 or 21 bases in length, there may be 6 or 10 probe bases, respectively, on each side of the SNP base, magnifying the impact of a single mismatch in the center. While longer capture probes, for example, up to about 31 bases or more, may be used, higher temperatures are preferably used with such probes. With capture probe lengths shorter than about 13 bases, there is a rapid decrease in specificity, with a shorter probe hybridizing to more than just the site of interest in the sample. Use of longer probes increases probe specificity. It is preferred to have an equal number of probe bases on each side of the SNP base.

Although the preferred SNP capture probe has a sequence of probe bases on each side of the SNP base, with at least four probe bases in each such sequence of probe bases, other probe configurations may be useful or necessary for specificity in some cases. Thus, SNP capture probes may have probe bases only on one side of the SNP base or may have fewer than four probe bases on one side of the SNP base, so long as the total length of the SNP capture probe is sufficient to bind to the SNP target when the SNP base of the SNP capture probe is complementary to the base at the SNP site in the target nucleic acid.

The invention utilizes an oxidation-reduction reaction between the mediator and guanines in the SNP target. After hybridization, the target nucleic acid hybridized to the probe attached to the electrode is reacted with a suitable mediator which is capable of oxidizing guanine in an oxidation-reduction reaction. To augment or increase the signal, additional labels, such as other nucleotide bases, including synthetic bases as are known in the art, such as 8-oxo-guanine and 8-oxo-adenine, could be also used, for example, if the target is amplified prior to conducting the hybridization. Alternatively, a detector sequence having guanine and/or other additional label, and that is complementary to a second sequence on the target may be used to enhance the signal from the target and increase sensitivity.

The ideal probe configuration to be used in a particular instance is adjusted to insure a positive signal from a specific SNP and to differentiate between a homozygote and a heterozygote for any given SNP. When any of the four bases may occur at a SNP site in the target (i.e., A, C, G, or T), or when it is not known which of these four bases may occur at the SNP site in the population, then four complementary probe sequences (four capture probes) and four electrodes are necessary for analysis of that SNP. Thus, the four probes used in the preferred embodiment of the invention are identical in base sequence except for the SNP base, which is at the site which corresponds to the SNP site where the determination of the presence of the polymorphism in the sample is to be made. Thus, each detection electrode has a unique capture probe with a different SNP base, A, C, G, or T. If the sample is homozygous, then only one electrode will produce a signal; however, if the sample is heterozygous, then two of the four electrodes will have a signal.

If there are three possible bases that may occur at a SNP site for all samples (for example, A, C or T, but not G), then three complementary probe sequences and three electrodes are necessary for the analysis of that specific SNP site. If the sample is homozygous, then only one electrode will produce a signal. If the sample is heterozygous, then two of the three electrodes will have a signal.

If it is known that there are only two bases that can occur at a particular SNP site for all samples (for example, A or C), then only two complementary probe sequences and two electrodes are necessary for analysis of that SNP. If the sample is homozygous, then only one electrode will produce an electrochemical signal. If the sample is heterozygous, then both electrodes will have a signal.

In the method of the invention herein, capture probes are prepared using knowledge of the normal genetic sequence on each side of a particular genetic site where one wishes to determine the presence of a SNP in a sample. Selection of sites for complementary probe sequences against specific targets preferably utilizes gene banks and probe analysis software. For example, if the nucleic acid that encodes synthesis of an enzyme normally has the following sequence (with an asterisk (*) indicating the single nucleotide polymorphism site of interest): 5′-T G C A T* G C A T-3′, this sequence would hybridize to 3′-A C G T A C G T A-5′. If there has been a mutation from T to C at the site of interest, yielding 5′-T G C A C* G C A T-3′, the sequence 3′-A C G T G C G T A-5′ would hybridize to the mutated nucleic acid. Using the technique of Thorp et al. discussed above for detecting nucleic acid hybridization, a different oxidation-reduction rate would be detected, depending on whether there was a mutation at the selected SNP site.

Because a single base mismatch in a central position in a nucleic acid containing a SNP can lower the melting temperature of the probe-target duplex by 8-20° C., the invention herein preferably utilizes elevated temperature to promote differential binding to the appropriate probe of the match DNA target over a mismatch target. Formamide, 20-50%, may also be included in the hybridization buffer to lower nonspecific binding of nucleic acid to the probes. The goal is to achieve a hybridization stringency of 100% for single nucleotide discrimination.

Electrodes and apparatus used for SNP detection. Preferably, in the method of the invention herein, the presence of hybridization is determined using the technology developed by Thorp et al. and by Xanthon, Inc. (Research Triangle Park, N.C.). This technology is disclosed and discussed in the patent of Thorp et al. (the '918 patent).

Basically, in the method used in the invention herein, a nucleic acid sample is contacted with a probe immobilized on an electrode to form a hybridized nucleic acid. The electrode itself may be entirely conductive or it may be in the form of a conductive working surface on a nonconductive substrate. Such electrodes are particularly useful in the above-discussed method of Thorp et al. and are useful in the invention herein. The probes are immobilized on the electrode by means known in the art. The primary immobilization layers that are preferably used in the invention herein comprise a) the polymer-electrode or b) an electrode having a self-assembled monolayer on the conductive working surface. Non-covalent mechanisms, such as direct adsorption to the electrode, may also be used.

For example, a polymer-electrode is disclosed in U.S. Pat. No. 5,968,745 of Thorp et al., the disclosure of which is incorporated herein by reference. This polymer-electrode comprises: (a) a substrate having a conductive working surface; and (b) a nonconductive outer polymer layer on said conductive working surface. The polymer layer has a plurality of microfluidic reaction openings distributed throughout the layer. A transition metal complex can transfer electrons through the layer to the conductive working surface. A probe is preferably bound to the polymer layer. This polymer layer can be brought into contact with the substrate at any point during treatment or reacting of the polymer. The polymer utilized in the polymer-electrode must be nonconductive and have openings therethrough. Preferred polymers are those derived from alkoxy silanes, such as isocyanato triethoxy silane. A polyethylene terephthalate (PET) membrane may also be used. These polymers have pores that extend generally perpendicularly from the surface through the film. The polymer layer may be placed in contact with the conductive working surface by any suitable means, such as by vacuum, by a liquid interface, by evaporation of a porous polymer film on the surface or by clamping the polymer layer to the surface.

Preferably, the sample solution containing the target nucleic acid is added to the polymer-electrode to which the probe is attached and hybridization carried out. After hybridization, the transition metal complex solution is added and a potential applied as in Thorp et al. (the '918 patent). Electrons from guanine are transferred to the conductive surface by the transition metal complex producing a detectable current.

Another example of a layer that may be used in the invention is the nonconductive self-assembled phosphonate monolayer of Eckhardt et al. (U.S. Pat. No. 6,127,127), the disclosure of which is incorporated herein by reference, which comprises phosphonate molecules, such as carboxy-alkyl phosphonate, having at the minimum at least one phosphonate group and at least one R₁ group. The R₁ group is covalently bound to a member of a binding pair. Generally, there is an organic spacer group, such as (CH₂)₁₁, located between the phosphonate group and the R₁ group. The transition metal complex can freely move through the self-assembled monolayer from reactants immobilized on the monolayer to the conductive working surface to transfer electrons to the conductive working surface. This electrode with the self-assembled monolayer is useful for the electrochemical detection of a label-bearing target in a sample. The self-assembled monolayer bound to the member of the binding pair is contacted with the sample, so that the immobilized member of the binding pair and the target if present form a target complex on the monolayer. The monolayer and the target complex, if present, are contacted with a transition metal complex that oxidizes the label-bearing target in an oxidation-reduction reaction between the transition metal complex and the label-bearing target, from which label-bearing target there is electron transfer to the transition metal complex. The oxidation-reduction reaction is detected at the electrode and the presence or absence of the nucleic acid is determined from the detected oxidation-reduction reaction. In some instances, amplification techniques as are known in the art may be used in conjunction with the invention.

The method of the invention herein preferably is performed on the microelectronic device of Thorp et al. (U.S. Pat. No. 6,132,971, the disclosure of which is incorporated herein by reference) in a plurality of wells, with one sample being tested per well. In this device, a 96-well plate with four detection electrodes per well (when there are four probes) may be used, plus the reference and counter electrodes. The oxidation-reduction rate is typically determined by measuring the electronic signal associated with the occurrence of the oxidation-reduction reaction. The electronic signal associated with the oxidation-reduction reaction may be measured by providing a suitable apparatus in electronic communication with the detection electrodes. A suitable apparatus is a potentiostat capable of measuring the electronic signal that is generated at each detection electrode so as to provide a measurement of the oxidation-reduction reaction rate of the reaction between the label in or on the captured target molecule and the mediator. The electronic signal may be characteristic of any electrochemical method, including cyclic voltammetry, normal pulse voltammetry, chronoamperometry, and square-wave voltammetry, with chronoamperometry and cyclic voltammetry being the currently preferred forms.

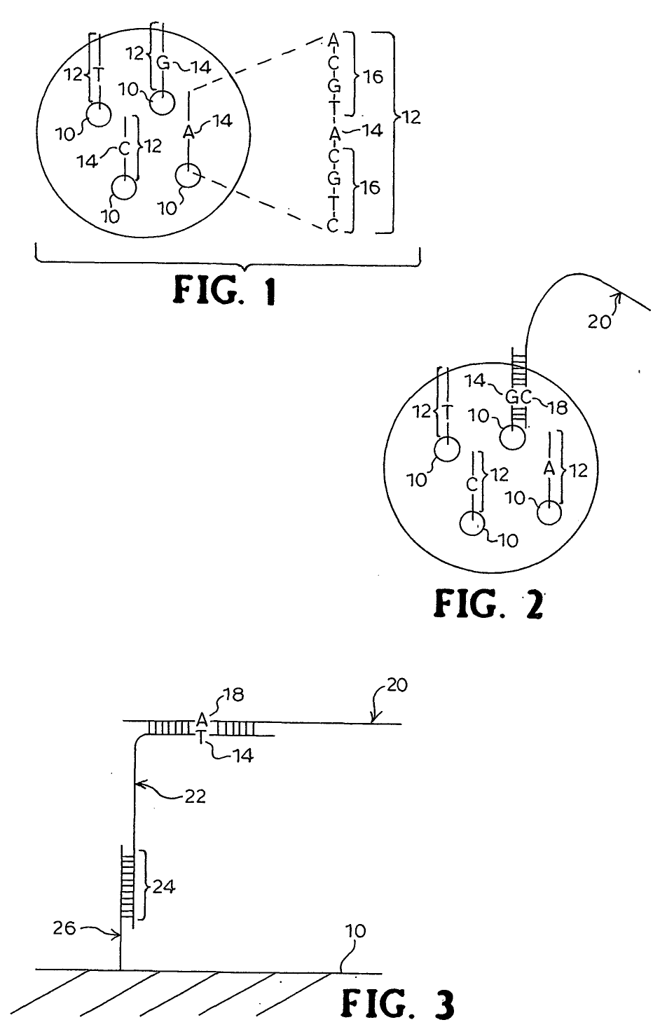

Schematic Representation of Invention. As shown in FIG. 1, the method of the invention for determining the presence or absence of a single nucleotide polymorphism at a selected SNP site in a selected nucleic acid sample utilizes a plurality of electrodes 10. Each SNP capture probe 12, comprising a SNP base 14 and adjacent probe bases 16 complementary to the normal bases comprising the sites adjacent the SNP site 18 is immobilized on each detection electrode 10 via a nonconductive layer (not shown). For a standard test there are four capture probe sequences having the G, C, A, and T variations substituted at the SNP base 14. A particular SNP capture probe 12 containing A as the SNP base 14 is shown in expanded form in FIG. 1. The other capture probes 12 in FIG. 1 would have the same adjacent probe bases 16 as shown in the expanded capture probe 12 and would only differ in the SNP base 14.

A nucleic acid target 20, when added to the electrodes 10, hybridizes to the SNP capture probe 12 where the SNP base 14 is complementary to the nucleotide at the SNP site 18 of the target 20. In FIG. 2, the SNP capture probe 12 that contains G as the SNP base 14 is shown as the complementary probe binding to the target 20.

Sandwich Assay. It is also useful in some applications to utilize a “sandwich” assay as part of the SNP detection method of the invention in order to improve hybridization efficiency (FIG. 3). In this method, there is a soluble probe sequence 22 that has bifunctional sequence specificity: one sequence is the SNP capture probe 12 and the second sequence 24 is complementary to a second capture probe 26 immobilized on the electrode 10. Thus, in practice, the SNP capture probe 12 portion of the soluble probe sequence 22 is allowed to hybridize to the nucleic acid target 20 (the SNP site 18 and the nucleotides adjacent the SNP site 18) in solution. The second sequence 24 of the soluble probe sequence 22 is then allowed to hybridize to the second capture probe 26, thereby immobilizing the target-soluble probe sequence complex onto the electrode, where it may be detected according to the invention herein. Only the soluble probe sequence 22 having a SNP base 14 complementary to the SNP site hybridizes to the target and provides a positive signal.

Biological sample. The samples used in the invention herein may be any biological sample, including, but not limited to, tissue samples such as biopsy samples and biological fluids such as whole blood, sputum, buccal swabs, urine and semen samples, bacterial cultures, soil samples, food samples, any other cell type or sample that contains DNA or other nucleic acid etc. The target nucleic acid may be of any origin, including animal, plant or microbiological (e.g., viral, prokaryotic, and eukaryotic organisms, including bacterial, protozoal, and fungal, etc.) depending on the particular purpose of the test. Examples include surgical specimens, specimens used for medical diagnostics, specimens used for genetic testing, environmental specimens, food specimens, dental specimens and veterinary specimens. Depending on the sample, it may only be necessary to lyse the cells, denature the DNA and hybridize to the probes. If required, genomic DNA may be purified from cells using commercially available kits. In some instances, for example, when RNA is present, the sample may need to be purified by techniques known in the art, so that the RNA does not interfere with the SNP detection in the DNA.

The sample may be processed or purified prior to carrying out the instant method in accordance with techniques known or apparent to those skilled in the art; and nucleic acids therein may be digested, fragmented, and/or amplified prior to carrying out the instant method, if so desired. Preferably the sample contains nucleic acid in a sufficient quantity so that amplification is not required; however, amplification may be utilized if desired to improve detection capability. If amplification is required, probes flanking the SNP site may be used, and an amplification method, such as PCR, employed.

The technique of the invention allows genetic analysis and correlation of simple and complex disease states to a specific SNP. SNPs can be used to predict an individual's susceptibility to disease, as well as to reveal an individual's response based on genetic variability to specific drugs for drug development and treatment strategies, allowing patient stratification based on their SNP profile. As the knowledge base of genetic functions of particular DNA-sites improves, there are many different beneficial uses for this information. For example, knowing the SNP characteristics of the persons on whom drugs are being tested allows determination of which drug(s) are beneficial, or have undesirable side-effects, for which type of patient(s), and knowing the SNP characteristics of patients being treated in a clinical setting allows refinement of the clinical treatment protocols.

The features of the present invention will be more clearly understood by reference to the following examples.

### EXAMPLES

### Example 1

Reagents and DNA. Inorganic reagents used in these experiments were analytical grade or higher. The source of the following reagents is as follows: carboxy-alkyl phosphonates made according to Example 2 or by Sigma Chemicals (St. Louis, Mo.) or Aldrich (Milwaukee, Wis.); [γ-³²P] adenosine triphosphate (ATP)(Pharmacia Biotech, Inc., Piscataway, N.J.); water (Milli-Q Plus purification system of Millipore, Bedford, Mass.); synthetic oligonucleotides (Oligos Etc., Inc., Wilsonville, Oreg. precoupled to the carboxy alkyl phosphonate; 1-Bromododecanoic acid, N,N′-dimethylformamide, and triethyl phosphite (Sigma); oxalyl chloride, dichloromethane, anhydrous ethanol, and triethylamine (Aldrich); and Na₂HPO₄, NaH₂PO₄, NaCl and conc. HCl (Fisher, Pittsburgh, Pa.).

### Example 2

Preferred Method of Preparation of Inmobilization Layers. Although certain phosphonic acids are currently commercially available, for example, amino propyl phosphonic acid and 2-carboxy ethyl phosphonic acid (Sigma or Aldrich), it is preferred to utilize a higher carbon phosphonic acid, such as a 11-carboxyundecane phosphonic acid (C-12 phosphonate). C-12 phosphonate and the self-assembled monolayer with phosphonate can be prepared as in U.S. Pat. No. 6,127,127. Polymer-electrodes can be prepared according to U.S. Pat. No. 5,968,745.

### Example 3

SNP Determination. In a preferred embodiment of the invention, genomic DNA of an individual donor is purified from 200 μl of blood. The average yield of total DNA is 6 μg, which has approximately 1.8×10⁶ copies of any one gene. This material is mechanically or enzymatically fragmented, denatured, added to the well of a 96-well microtiter plate, and allowed to hybridize to the capture probes using the method of Thorp et al. (U.S. Pat. No. 5,871,918). A schematic diagram of the structure of the capture probes for such a determination is shown in FIG. 1. If the donor is homozygous for the sequence tested both copies of the gene are identical. As shown in FIG. 2, in this case, only one electrode with the complementary sequence has target DNA hybridized to it and generates an electrochemical signal. The other three electrodes do not have any hybridized material, and consequently, have no signal. If the donor is heterozygous for the sequence tested, two of the four electrodes have hybridized material and give an electrochemical signal. The remaining electrodes do not have any signal. In this manner, the SNPs for a sequence can be determined, as well as whether the donor is homozygous or heterozygous at this allele.

While the invention has been described with reference to specific embodiments, it will be appreciated that numerous variations, modifications, and embodiments are possible, and accordingly, all such variations, modifications, and embodiments are to be regarded as being within the spirit and scope of the invention.

1. A method of determining the presence or absence of a single nucleotide polymorphism at a selected SNP site in a target nucleic acid, comprising:
a) obtaining information about base sequences adjacent to the selected SNP site;
b) providing a plurality of SNP capture probes, each of said SNP capture probes comprising a SNP base having a sequence of probe bases on at least one side of said SNP base, each said sequence of probe bases being complementary to a corresponding base sequence adjacent the selected SNP site, each of said SNP capture probes having a different SNP base, wherein the length of each of the SNP capture probes is sufficient so that the SNP capture probe having the SNP base that is complementary to the base at the SNP site in the target nucleic acid binds to the SNP target nucleic acid;
c) providing an electrode for each SNP capture probe, each of said electrodes having a non-conductive immobilization layer on a conductive working surface of a substrate;
d) immobilizing each SNP capture probe on its immobilization layer on its electrode;
e) contacting each electrode with the target nucleic acid;
f) contacting the target nucleic acid with a transition metal complex that oxidizes guanine in an oxidation-reduction reaction under conditions that cause an oxidation-reduction reaction between the transition metal complex and guanine, wherein there is electron transfer from guanine to the transition metal complex, resulting in regeneration of the reduced form of the transition metal complex as part of a catalytic cycle;
g) detecting whether there is hybridization between each SNP capture probe and the target nucleic acid, by detecting the oxidation-reduction reaction at each electrode; and
h) using the hybridization between each of the plurality of SNP capture probes and the target nucleic acid to determine whether the target nucleic acid has a single nucleotide polymorphism at the selected SNP site.

2. The method according to claim 1, wherein the nonconductive immobilization layer
comprises a self-assembled monolayer.

3. The method according to claim 1, wherein the nonconductive immobilization layer comprises a polymer.

4. The method according to claim 1, wherein each of the capture probes comprises a neutral backbone.

5. The method according to claim 4, wherein the neutral backbone is selected from the group consisting of peptide backbones, p-ethoxy backbones and morpholine backbones.

6. The method according to claim 1, wherein each of the capture probes has a sugar-phosphate backbone.

7. The method according to claim 6, wherein the capture probe is an oligonucleotide.

8. The method according to claim 1, wherein there are four SNP capture probes.

9. The method according to claim 1, wherein three different bases are possible at the SNP site and there are three SNP capture probes.

10. The method according to claim 1, wherein two different bases are possible at the SNP site and there are two SNP capture probes.

11. The method according to claim 1, wherein each of the SNP capture probes has a sequence of probe bases on each side of said SNP base.

12. The method according to claim 11, wherein the sequence of probe bases on at least one side of the SNP base comprises at least four probe bases.

13. The method according to claim 1, wherein the sample is selected from the group consisting of: synthetic or natural oligonucleotides, surgical specimens, specimens used for medical diagnostics, specimens used for genetic testing, environmental specimens, food specimens, dental specimens and veterinary specimens.

14. The method according to claim 1, wherein each capture probe is immobilized on an electrode through a covalent bond to a silane molecule.

15. The method according to claim 1, wherein each capture probe is immobilized on an electrode through a covalent bond to a phosphonate molecule.

16. The method according to claim 1, wherein the immobilization layer comprises the immobilized capture probe.

17. The method according to claim 1, wherein the capture probe comprises 9-31 bases.

18. The method according to claim 1, wherein each SNP capture probe is on a soluble probe sequence that also comprises a second sequence that is complementary to a second capture probe immobilized on the electrode.

19. The method according to claim 18, wherein each of the SNP capture probes has a sequence of probe bases on each side of said SNP base.

20. The method according to claim 18, wherein the sequence of probe bases on at least one side of the SNP base comprises at least four probe bases.

21. The method according to claim 18, wherein the capture probe comprises 9-31 bases.

22. A method of determining the presence or absence of a single nucleotide polymorphism at a selected SNP site in a target nucleic acid, comprising:
a) providing a plurality of SNP capture probes, each of said SNP capture probes comprising a SNP base having a sequence of probe bases on at least one side of said SNP base, each said sequence of probe bases being complementary to a corresponding base sequence adjacent the selected SNP site, each of said SNP capture probes having a different SNP base, wherein the length of each of the SNP capture probes is sufficient so that the SNP capture probe having the SNP base that is complementary to the base at the SNP site in the target nucleic acid binds to the SNP target nucleic acid;
b) contacting each electrode with the target nucleic acid;
c) contacting the target nucleic acid with a transition metal complex that oxidizes guanine in an oxidation-reduction reaction under conditions that cause an oxidation-reduction reaction between the transition metal complex and guanine, wherein there is electron transfer from guanine to the transition metal complex, resulting in regeneration of the reduced form of the transition metal complex as part of a catalytic cycle;
d) detecting whether there is hybridization between each SNP capture probe and the target nucleic acid, by detecting the oxidation-reduction reaction at each electrode; and
e) using the hybridization between each of the plurality of SNP capture probes and the target nucleic acid to determine whether the target nucleic acid has a single nucleotide polymorphism at the selected SNP site.

23. The method according to claim 22, wherein the nonconductive immobilization layer comprises a self-assembled monolayer.

24. The method according to claim 22, wherein the nonconductive immobilization layer comprises a polymer.

25. The method according to claim 22, wherein each of the capture probes comprises a neutral backbone.

26. The method according to claim 25, wherein the neutral backbone is selected from the group consisting of peptide backbones, p-ethoxy backbones and morpholine backbones.

27. The method according to claim 22, wherein each of the capture probes has a sugar-phosphate backbone.

28. The method according to claim 27, wherein the capture probe is an oligonucleotide.

29. The method according to claim 22, wherein there are four SNP capture probes.

30. The method according to claim 22, wherein three different bases are possible at the SNP site and there are three SNP capture probes.

31. The method according to claim 22, wherein two different bases are possible at the SNP site and there are two SNP capture probes.

32. The method according to claim 22, wherein each of the SNP capture probes has a sequence of probe bases on each side of said SNP base.

33. The method according to claim 32, wherein the sequence of probe bases on at least one side of the SNP base comprises at least four probe bases.

34. The method according to claim 22, wherein the sample is selected from the group consisting of: synthetic or natural oligonucleotides, surgical specimens, specimens used for medical diagnostics, specimens used for genetic testing, environmental specimens, food specimens, dental specimens and veterinary specimens.

35. The method according to claim 22, wherein each capture probe is immobilized on an electrode through a covalent bond to a silane molecule.

36. The method according to claim 22, wherein each capture probe is immobilized on an electrode through a covalent bond to a phosphonate molecule.

37. The method according to claim 22, wherein the immobilization layer comprises the immobilized capture probe.

38. The method according to claim 22, wherein the capture probe comprises 9-31 bases.

39. The method according to claim 22, wherein each SNP capture probe is on a soluble probe sequence that also comprises a second sequence that is complementary to a second capture probe immobilized on the electrode.

40. The method according to claim 39, wherein each of the SNP capture probes has a sequence of probe bases on each side of said SNP base.

41. The method according to claim 39, wherein the sequence of probe bases on at least one side of the SNP base comprises at least four probe bases.

42. The method according to claim 39, wherein the capture probe comprises 9-31 bases.
